(19) United States
(12) Patent Application Publication
(10) Pub. No.: US 2011/0160619 A1
(43) Pub. Date: Jun. 30, 2011

(54) METHOD AND APPARATUS FOR A SCENTED ALARM CLOCK BASED ON SLEEP STATE

(75) Inventor: Asher Gabara, Murray Hill, NJ (US)

(73) Assignee: LCtank LLC

(21) Appl. No.: 12/651,279

(22) Filed: Dec. 31, 2009

(51) Int. Cl. A61B 5/11; A61M 21/00; G04B 47/00

(52) U.S. Cl. 600/595; 600/27; 368/10

(57) ABSTRACT

A method and apparatus for affecting the dreams of the subject or individual is presented. The apparatus first predicts or detects the desired sleep state and presents a scent to the subject. The apparatus then awakens the subject after a given period of time using external stimulus. The desired sleep state may be the REM sleep state, and the period of time may be one minute. Upon awakening, the subject will recall dreams that have been affected by the emitted scent. If the scent is pleasant, then the subject will recall having pleasant dreams.

### BACKGROUND OF THE INVENTION

Sleep can have several states; some are the “light sleep state”, “deep sleep state” and an REM (Rapid Eye Movement) state. Being awoken from these different states can influence the state of the awoken sleeper. Ideally, once being awoken from a sleep, a desirable situation would be to wake in a pleasant state from a pleasant dream. In a paper by RebecCa Carroll of the National Geographic News “Smells Influence Dreams, Study Says,” Boris Stuck of the University Hospital Mannheim in Germany has shown that when a scent is smelled by a person in REM sleep, one minute prior to waking, that the person's dreams will be affected. When the scent is positive, such as that of a rose, the person will recall having positive dreams.

There are several existing devices that have the intention of affecting the user's sleep and dreams. One such device, the Sleeptracker (manufactured by Innovative Sleep Solutions, Inc., of Atlanta, Ga., USA), is worn on the wrist and wakens the user during light sleep, which is determined by tracking the movement of the user's body.

Stanford University holds U.S. Pat. No. 6,468,234, entitled “SleepSmart”, and was published on 22 Aug. 1998. This document relates to a method and apparatus for measuring sleep quality that utilizes pressure and temperature sensors incorporated in a sheet, which can be laid on top of a conventional mattress. The apparatus comprises one or more layers of arrays of integrated pressure and/or temperature sensor pads for collecting data relating to physical properties such as an individual's position, temperature, sound, vibration and movement.

Brown University has US patent application number 2006/0293608 that was published on Dec. 28, 2006. This application describes a device and a method for waking a user in a desired sleep state. The device may predict an occurrence when the user will be in the desired sleep state, such as light sleep, and wake the user during that predicted occurrence.

This device, by Axon Sleep Research Laboratories (Providence, R.I., USA) is an intelligent alarm clock (also called “SleepSmart”) that monitors sleep cycles and attempts to awaken the user at an optimal point in the sleep cycle. It appears that the name “SleepSmart” is used by both the Stanford and Axon's references but refer to different aspects of sleep. SleepSmart in Axon requires the user to sleep with a headband that measures physiological data. It has also been suggested that sleep staging can be obtained from respiration and heart rate information during sleep.

Another device is the aXbo Sleep Phase Alarm Clock which is a wristband that reads body movements. In addition, a Wake n' Bacon is a device that starts cooking bacon ten minutes before wake-up time. There are also devices that awake the users by light rather than by sound.

### BRIEF SUMMARY OF THE INVENTION

The aforementioned effect is the central idea behind an alarm clock that will emit a scent one minute prior to going off, in order to affect the dreams of the user.

The proposed device can awake the user while the user is in the dream-heavy REM state. This is in fact the more natural state to be awoken. Dr. Gregory Belenky, a sleep expert at Washington State University Spokane, said light stage sleep isn't the best time to be woken by an alarm. He says that you are instead most likely to wake up out of a dream, and that “(a light sleep stage) is the hardest time to wake a person up unnaturally.”

One minute prior to the alarm, a scent will be emitted by the device. If the scent is pleasant, then the dreams of the user will be pleasant. At alarm time, the user will be awakened, either by sound, by light, or by both.

### BRIEF DESCRIPTION OF THE DRAWINGS

Please note that the drawings shown in this specification may not be drawn to scale and the relative dimensions of various elements in the diagrams are depicted schematically and not to scale.

FIG. 1 shows a person sleeping.

FIG. 2 illustrates a positive scent being given to the person.

FIG. 3 depicts a negative scent being given to the person.

FIG. 4 shows an sleep state detector that can determine whether the person is in REM sleep.

FIG. 5 depicts the flowchart of the waking up sequence of this inventive technique.

FIG. 6 shows an sleep state detector leading to the emission of a scent.

FIG. 7 depicts an sleep state detector using the stimulus of light to awake the person of this inventive technique after the scent is emitted.

FIG. 8 depicts an sleep state detector using the stimulus of sound to awake the person of this inventive technique after the scent is emitted.

### DETAILED DESCRIPTION OF THE INVENTION

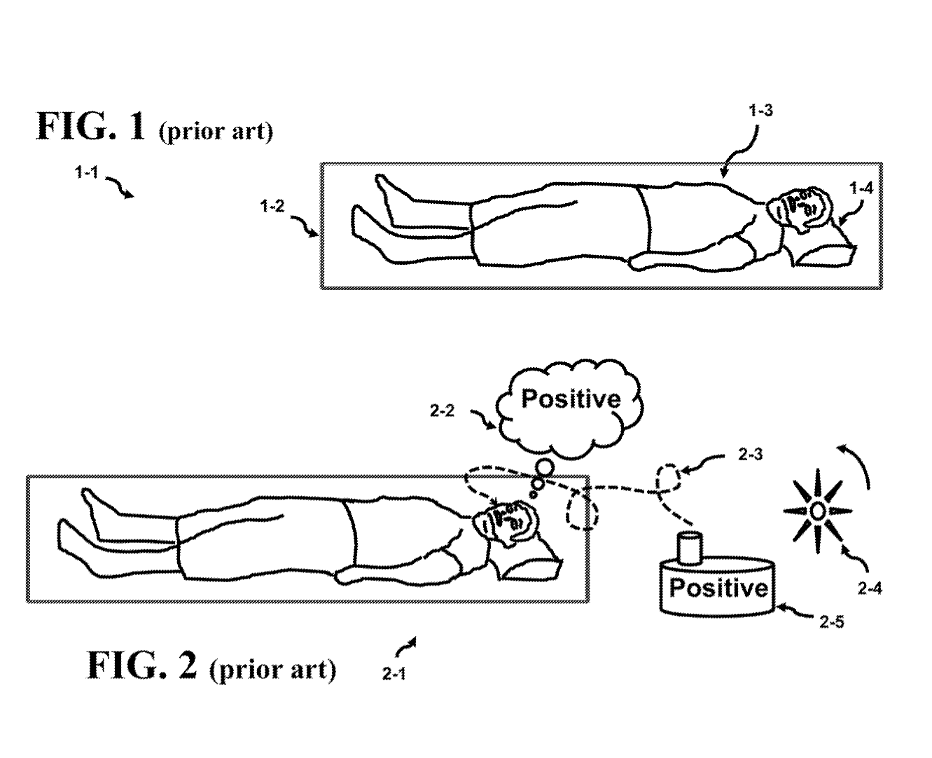

FIG. 1 illustrates a view 1-1 of a typical subject 1-3 sleeping on a bed 1-2. The subject's head in on a pillow 1-4. This is the approximate position and posture of a typical subject sleeping.

A positive scent 2-3, hie that of a rose, candy, perfume, etc. can be extracted from a container 2-5 as depicted in FIG. 2. This view 2-1 shows the subject having positive dreams 2-2 after inhaling the positive scent 2-3 during an REM sleep state. A spinning fan 2-4 diffuses or directs the scent towards the subject.

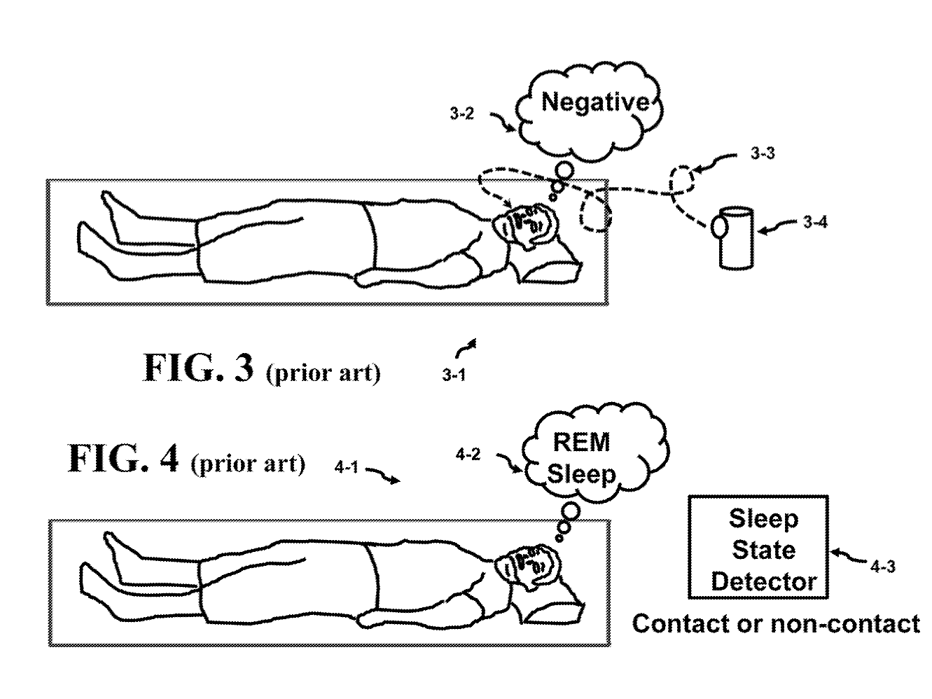

A negative scent 3-3, like that of a rotten egg, spoiled meat, waste products, etc. can be extracted from a aerosol can 3-4 as depicted in FIG. 3. This view 3-1 shows the subject having negative dreams 3-2 after inhaling the negative scent 3-3 during an REM sleep state. Unlike before, an aerosol can 32 is used as an alternative way to diffuse or direct the scent towards the subject.

A system 4-1 to detect when the subject is in REM sleep 4-2 is illustrated in FIG. 4. The sleep state detector 4-3 may need to be in contact with the subject; however, non-contact techniques can be performed. For example, in patent application number 2007/0118054 by EarlySense, their methods employ sensors, for example, non-contact sensors, adapted to detect vital signs, such as heart rate or respiration rate, to produce signals that can be analyzed for trends, for deviations, or for comparison to prior conditions or criteria.

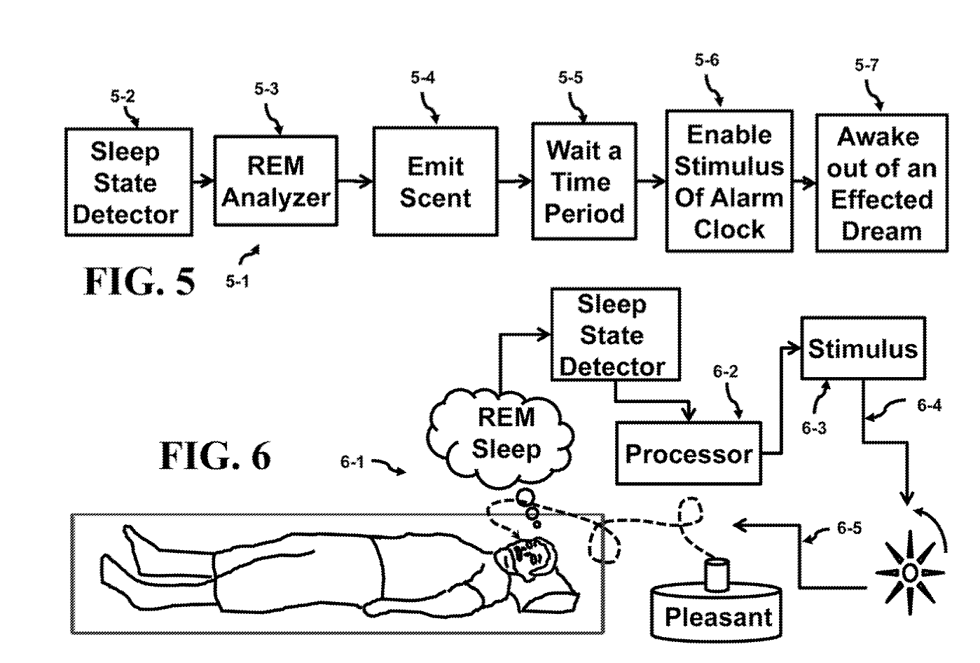

FIG. 5 illustrates a flowchart 5-1 showing one embodiment of the invention. The sleep state detector 5-2 feeds collected information to the REM analyzer 5-3. The REM analyzer may be coupled to a processor (not shown) to extract statistical data. If REM sleep is detected and it is the appropriate duration of time before the stimulus of the alarm, a scent 5-4 is emitted; a period of time 5-5 is used for the scent to react on the subject. After this period of time has elapsed, the stimulus of the alarm clock 5-6 is enabled. This stimulus awakens the subject out of a dream affected by the scent 5-7.

The sleep state detector in FIG. 6 can be applied to a processor 6-2 that issues a stimulus 6-3 to control 6-4 the fan that displaces 6-5 the scent to the person. The spinning fan diffuses the scent towards the subject. The person after inhaling the scent during an REM sleep state will start having dreams affected by the scent.

FIG. 7 depicts an added stimulus control 7-2 that enables the lights 7-3 that are applied 7-4 to the subject. The lights 7-3 are enabled after the subject has had an opportunity to have dreams affected by the emitted scent.

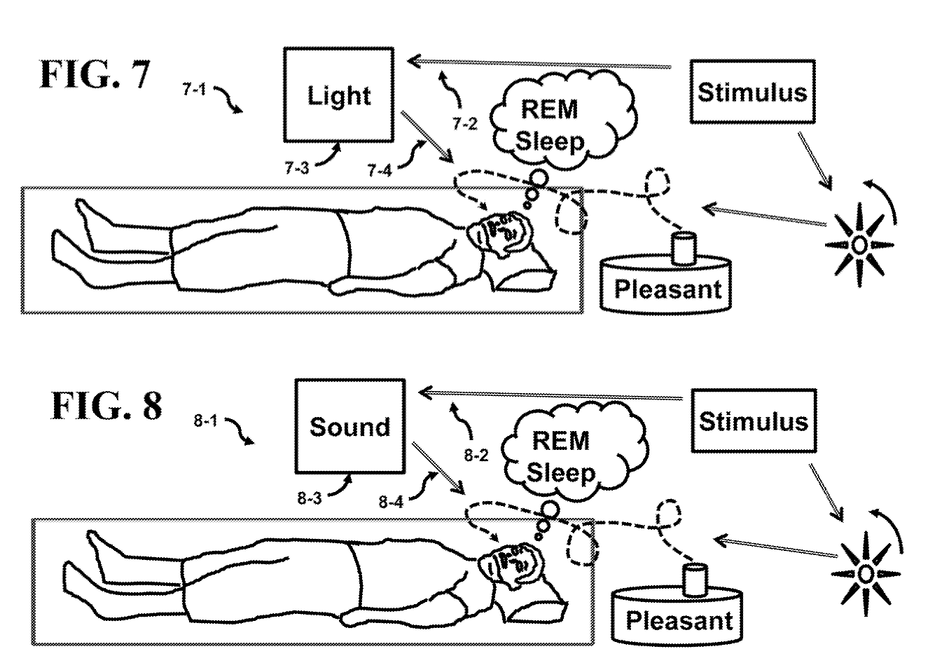

FIG. 8 depicts a different added stimulus control 8-2 that enables a sound 8-3 that is applied 8-4 to the subject. The sound 8-3 is enabled after the subject has had an opportunity to have dreams affected by the emitted scent.

It is understood that the above description are only illustrative of the principle of the current invention. It is understood that the various embodiments of the invention, although different, are not mutually exclusive. In accordance with these principles, those skilled in the art may devise numerous modifications without departing from the spirit and scope of the invention. For example, the sound in FIG. 8 can be applied to the subject using personal headphones or a large speaker positioned on a shelf. Also, both the lights and the sound can be applied to the subject during the stimulus period.

What is claimed is:

1. An alarm clock comprising:
a sleep state detector coupled to a subject to determine when said subject is in a given sleep state;
a scent applied to said subject in said given sleep state;
a processor coupled to said sleep state detector to determine when to apply at least one stimulus to awaken said subject.

2. The apparatus of claim 1, whereby
said given sleep state is a dream-heavy REM (Rapid Eye Movement) state.

3. The apparatus of claim 1, whereby
said stimulus is a way to awaken said subject selected from the group consisting of light, sound, touch and movement.

4. The apparatus of claim 1, whereby
said processor calculates a time period.

5. The apparatus of claim 4, whereby
said time period is one minute.

6. The apparatus of claim 3, whereby
a second stimulus is selected from said group.

7. The apparatus of claim 1, whereby
said scent creates undesirable dreams.

8. The apparatus of claim 1, whereby
said scent creates pleasant dreams.

9. The apparatus of claim 1, whereby
said subject is awoken in a preferred state.

10. A method of waking a subject comprising the steps of:
applying a scent to said subject sleeping in a given sleep state;
waiting a first time period; and
waking said subject.

11. The method of claim 10, further comprising the steps of:
a sleep state detector coupled to said subject to determine when said subject is in said given sleep state.

12. The method of claim 11, wherein
said given sleep state is a dream-heavy state.

13. The method of claim 11, further comprising the steps of:
a processor coupled to said sleep state detector to calculate when to apply at least one stimulus.

14. The method of claim 13, wherein
said waking occurs by the application of said at least one stimulus to awaken said subject.

15. The method of claim 14, wherein
said stimulus is a way to awaken said subject selected from the group consisting of light, sound, touch and movement.

16. The method of claim 10, wherein
said subject is placed into a preferred state.

17. The method of claim 10, wherein
said scent creates pleasant dreams.

18. The method of claim 10, wherein
said scent creates undesirable dreams.

19. An alarm clock comprising:
a sleep state detector coupled to a subject;
a scent being applied to said subject in a sleep state; and
a processor coupled to said sleep state detector to determine when to apply at least one stimulus to awaken said subject.

20. The apparatus of claim 19, whereby
said sleep state is the dream-heavy REM (Rapid Eye Movement) state.
